(19) United States
(12) Patent Application Publication
(10) Pub. No.: US 2006/0276699 A1
(43) Pub. Date: Dec. 7, 2006

(54) Spectral probe for blood vessel diagnosis

(75) Inventors: Yuichi Komachi, Saitama (JP); Hidetoshi Sato, Saitama (JP); Hideo Tashiro, Saitama (JP)

Correspondence Address: Reed Smith LLP, Suite 1400, 3110 Fairview Park Drive, Falls Church, VA, 22042-4503, US

(73) Assignee: RIKEN and MACHIDA ENDOSCOPE CO., LTD.

(21) Appl. No.: 11/378,311

(22) Filed: Mar. 20, 2006

(30) Foreign Application Priority Data
May 13, 2005 (JP) 2005-141059

(51) Int. Cl. A61B 5/00

(52) U.S. Cl. 600/341

(57) ABSTRACT

A spectral probe for blood vessel diagnosis that can be used for diagnosing blood vessels with high reliability, without imposing an excessive burden on patients. A Raman spectrum measuring probe and either an endoscope or an ultrasonic probe are integrated. A mechanism is provided for bringing the tip of the Raman probe into contact with an affected area so as to eliminate the influence of blood when measuring. An endoscope has a forward-looking view in the blood vessel, while the Raman probe is of the lateral-view type for facilitating the measurement of blood vessel walls. A fixing balloon is provided at the probe tip portion that is inserted into a blood vessel, and a window for the Raman probe is provided on the opposite side of the balloon. As the fixing balloon is inflated, part of it comes into contact with the internal wall of a blood vessel and fixes the probe, while leaving a gap between the internal wall of the blood vessel and the probe. The window comes into contact with the affected area. Because the blood is allowed to flow through the gap, the blood stream is not stopped even when the balloon is inflated.

### CLAIM OF PRIORITY

The present application claims priority from Japanese application JP 2005-141059 filed on May 13, 2005, the content of which is hereby incorporated by reference into this application.

### BACKGROUND OF THE INVENTION

1. Field of the Invention

The present invention relates to a spectral probe and in particular to a spectral probe for blood vessel diagnosis that can be used for diagnosing arterial sclerosis and the like by inserting the probe into a blood vessel.

2. Background Art

Anal. Chem., 72, 3771-3775 (2000) and JP Patent Publication (Kokai) No. 2004-294109A disclose spectral probes in which optical fibers are employed in an optical path for guiding excitation light from a light source to a measured portion and in an optical path for guiding Raman scattered light emitted from the measured portion to a light-receiving portion.

Patent Document 1: JP Patent Publication (Kokai) No. 2004-294109 A

Non-Patent Document 1: Anal. Chem., 72, 3771-3775 (2000)

### SUMMARY OF THE INVENTION

In diagnosing arterial sclerosis, it is necessary to measure cholesterol accumulated on the outside of a blood vessel wall. However, accumulated plaques on the outside of the blood vessel wall cannot be measured with an intravascular endoscope or a vascular echo. Consequently, it is conceivable to judge the condition of plaques accumulated on the outside of the blood vessel wall using Raman scattering by irradiating an affected area with light from a probe inserted into the blood vessel. Because the probe is inserted into the blood vessel, excitation light is transmitted via an optical fiber, and light reception is also performed via an optical fiber.

However, conventional spectral probes are comprised of optical fibers alone that transmit and receive light. Therefore, although the probe can be inserted into a blood vessel, its application to the diagnosis of blood vessels is problematic. First, the location of the probe tip cannot be confirmed. Therefore, for guiding the probe to an affected area in order to confirm the diagnosed area, an imaging device for external X-ray irradiation needs to be used, thereby increasing cost. Also, because the spectral probe cannot be fixed in the blood vessel, the target affected area cannot be reliably measured. Furthermore, the measurement is subject to the interference of blood in the blood vessel and is therefore difficult.

It is an object of the invention to solve such problems and provide a spectral probe for blood vessel diagnosis that can be used for diagnosing blood vessels with high reliability, without imposing an excess burden on patients.

In accordance with the invention, a Raman spectrum measuring probe and either an endoscope or an ultrasonic probe are integrated. A mechanism is provided for bringing the tip of the Raman probe in contact with an affected area so as to eliminate the interference of blood during measurement. The endoscope has a forward-looking view in a blood vessel, and the Raman probe is of the lateral-view type for facilitating the measurement of blood vessel walls. A fixing balloon is provided at the probe tip portion that is inserted into the blood vessel, as a mechanism for causing the probe tip to come into contact with the affected area. The mechanism is structured such that when the fixing balloon is inflated, it does not occupy the entire internal walls of the blood vessel. Instead, part of the balloon comes into contact with the internal wall of the blood vessel when the probe is fixed, thereby forming a gap between the internal walls of the blood vessel and the probe. Because blood is allowed to flow through the gap, the blood stream is not blocked even when the balloon is inflated. A window for the Raman probe is provided on the opposite side of the balloon. As the fixing balloon is inflated, the window comes into contact with the affected area. Therefore, diagnoses can be made without the interference of blood during measurement and without imposing an excess burden on the human body.

An embodiment of the spectral probe for blood vessel diagnosis of the invention includes a Raman probe portion comprising: a long member inserted into a blood vessel; a light-transmitting window provided on the side wall of the tip of the long member; a balloon provided in the side wall of the tip portion of the long member opposite to the light-transmitting window; an optical fiber for guiding light from a light source to the light-transmitting window; and an optical fiber for guiding Raman scattered light that enters the long member via the light-transmitting window to the outside, a tube for introducing a fluid into the balloon, an endoscope portion comprising an optical fiber for guiding illumination light to the tip of the long member and an image fiber. The probe also includes a tube for injecting normal saline solution into an area that is irradiated with the illumination light from the endoscope portion.

Another embodiment of the spectral probe for blood vessel diagnosis of the invention includes a Raman probe portion comprising: a long member that is inserted into a blood vessel; a light-transmitting window provided in the side wall of the tip of the long member; a balloon provided in the side wall of the tip of the long member opposite to the light-transmitting window; an optical fiber for guiding light from a light source to the light-transmitting window; and an optical fiber for guiding Raman scattered light that enters the long member via the light-transmitting window to the outside. The probe also includes a tube for introducing a fluid into the balloon, and an ultrasonic probe for obtaining an ultrasonic image of an area at the tip portion of the long member.

In accordance with the invention, the Raman probe tip can be guided to the affected area in a blood vessel with ease and fixed there without interfering with the blood stream, whereby the Raman spectra of the affected area can be measured without the interference of blood.

### BRIEF DESCRIPTION OF THE DRAWINGS

FIG. 1 shows an overall view of an embodiment of a spectral probe for blood vessel diagnosis according to the invention.

FIG. 2 shows a diagram of the connection between the spectral probe for blood vessel diagnosis and external devices.

FIG. 3 shows a schematic perspective view of the structure of the tip of an effective portion of the spectral probe for blood vessel diagnosis.

FIG. 4 shows a schematic cross-section of the tip of the effective portion of the spectral probe for blood vessel diagnosis.

FIGS. 5A and 5B show a schematic diagram of a balloon being inflated in a blood vessel during measurement.

FIGS. 6A and 6B show a schematic view of the structure of the tip of a Raman probe portion.

FIG. 7 shows a graph of measurement results.

FIG. 8 shows an overall view of another embodiment of the spectral probe for blood vessel diagnosis according to the invention.

FIG. 9 shows a diagram of the connection between the spectral probe for blood vessel diagnosis and the outside.

FIG. 10 shows a schematic perspective view of the structure of the tip of the effective portion of the spectral probe for blood vessel diagnosis.

FIG. 11 shows a schematic cross-section of the tip of the effective portion of the spectral probe for blood vessel diagnosis.

FIGS. 12A and 12B show a schematic diagram of the balloon being inflated in a blood vessel during measurement.

### DETAILED DESCRIPTION OF THE INVENTION

Hereafter, an embodiment of the invention will be described with reference to the drawings.

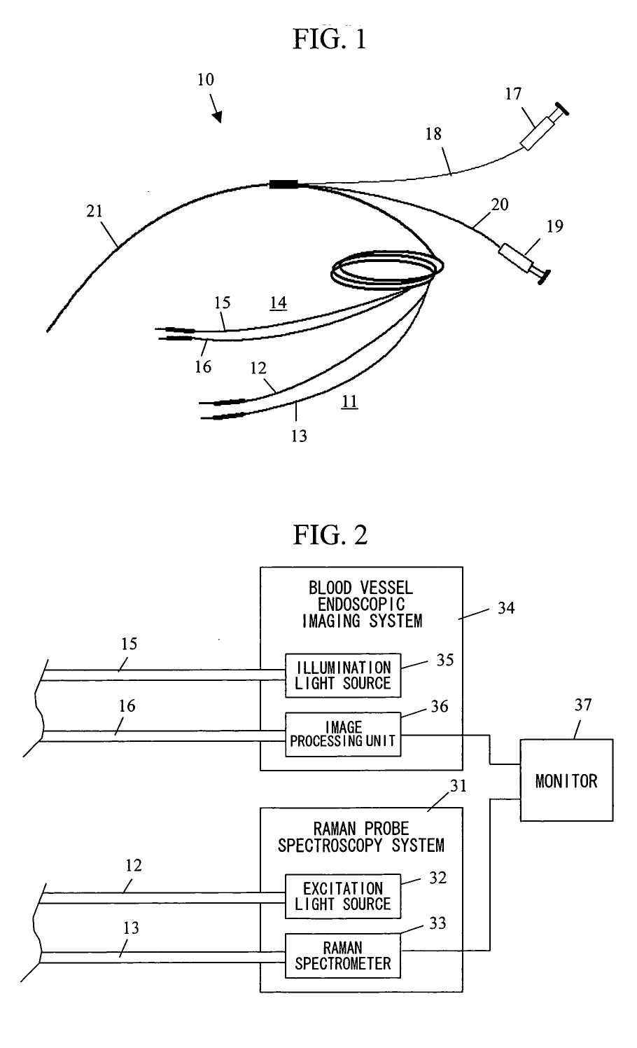

FIG. 1 shows an overall view of an embodiment of the spectral probe for blood vessel diagnosis according to the invention.

This spectral probe for blood vessel diagnosis 10 comprises a Raman probe portion 11 comprising an excitation-light optical fiber 12 and a light-receiving optical fiber 13, an endoscope portion 14 comprising a light guide 15 for guiding illumination light and an image fiber 16 for transmitting an image, a tube 18 connected to a syringe 17 for a blood-removing solution, and a tube 20 connected to a balloon syringe 19. The overall length of the spectral probe for blood vessel diagnosis 10 is 3 m, and the length of an effective portion 21 that can be inserted into a blood vessel is 1.5 m. The diameter of the effective portion 21 is 2 mm for making measurement in an artery possible.

FIG. 2 shows a diagram of the connection between the spectral probe for blood vessel diagnosis and external devices. The excitation-light optical fiber 12 and the light-receiving optical fiber 13 of the Raman probe portion 11 are connected to an excitation light source 32 and a Raman spectrometer 33, respectively, of a Raman probe spectroscopy system 31. A bandpass filter for eliminating Raman scattered light that develops in the optical fibers is mounted at the tip of the excitation-light optical fiber. An edge filter for cutting off Rayleigh scattered light from an affected area is mounted at the tip of the light-receiving optical fiber. As a light source, near-infrared light is employed, which causes the human body to produce little fluorescence that constitutes a disturbing light when measuring Raman scattering. The excitation-light optical fiber guides near-infrared light from a titanium-sapphire laser to an affected area. The optical path is perpendicularly bended by a mirror at the tip such that a blood vessel wall can be irradiated with laser light perpendicularly. The scattered light produced at the affected area is guided to the spectroscope through the light-receiving fiber.

The light guide 15 and the image fiber 16 of the endoscope portion 14 are connected to an illumination light source 35 and an image processing unit 36, respectively, of a blood vessel endoscope imaging system 34. An image of the inside of a blood vessel acquired via the image fiber 16 of the endoscope portion 14 is received and processed by a CCD provided in the image processing unit 36 and then displayed on a monitor 37. The monitor 37 also displays the Raman scattering spectra of the affected area obtained by the Raman spectrometer 33.

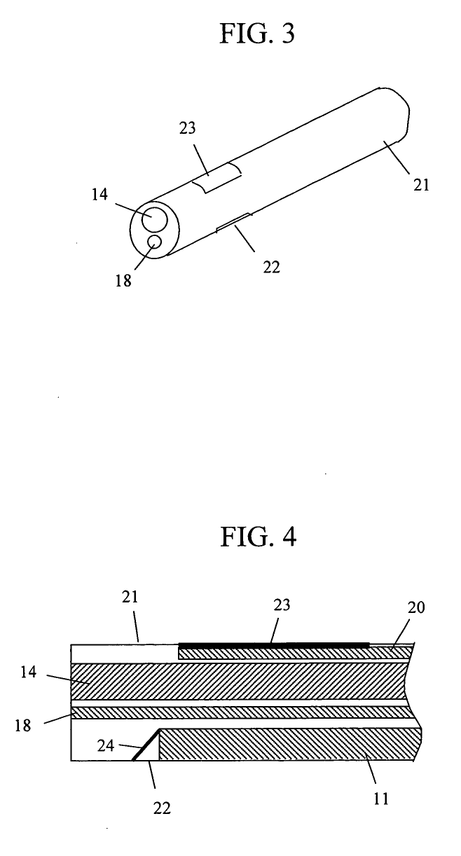

FIG. 3 shows a schematic perspective view of the structure of the tip of the effective portion of the spectral probe for blood vessel diagnosis according to the embodiment, and FIG. 4 shows its schematic cross-section.

On the tip of the effective portion 21 that is inserted into a blood vessel, there are exposed the tip of the endoscope 14 and the tip of the tube 18, which is an outlet for normal saline solution for ensuring a field of view for the endoscope. A Raman measurement window 22 is provided on the side of the effective portion 21, and a balloon 23 is provided on the side of the effective portion opposite to the Raman measurement window 22. The balloon 23 is designed to be inflated by injecting normal saline solution from the balloon syringe 19 via the tube 20. As shown in FIG. 4, the excitation light from the Raman probe 11 is reflected by a mirror 24 provided in the effective portion 21, and then shone against the blood vessel wall through the Raman measurement window 22. Raman scattered light from the blood vessel wall as it is irradiated with the excitation light is passed through the Raman measurement window 22, reflected by the mirror 24, and then introduced into the Raman probe 11.

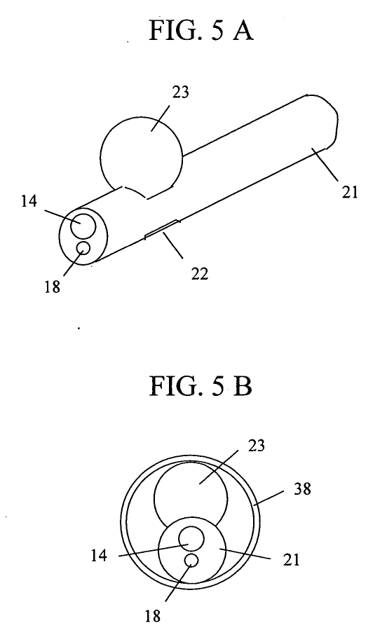

FIGS. 5A and 5B show a schematic diagram of the balloon being inflated in a blood vessel during measurement. FIG. 5A shows a perspective view of the tip of the effective portion of the spectral probe for blood vessel diagnosis, and FIG. 5B shows a schematic cross-sectional view of the blood vessel, as seen from the probe tip.

The balloon 23 has the function of fixing the effective portion 21 of the spectral probe for blood vessel diagnosis 10 at a desired location in a blood vessel 38, and the function of bringing the measurement window 22 of the Raman probe into contact with an affected area. An operator inserts the spectral probe for blood vessel diagnosis into a blood vessel, and, while monitoring the condition of internal walls of the blood vessel via the image obtained by the endoscope portion 14 and displayed on the monitor 37, guides the probe tip to the affected area, where he or she determines the measurement location. At this time, the blood at the probe tip that hinders image acquisition is removed by operating the syringe 17 in order to cause the normal saline solution to flow from the tube 18 exposed at the probe tip, thereby ensuring a field of view.

Once the measurement location is determined, the normal saline solution is injected into the balloon 23 from the balloon syringe 19, so that the balloon 23 is inflated for fixing the probe tip at the measurement location. Then, as shown in FIG. 5B, the balloon 23 comes into contact with the internal wall of the blood vessel 38, and causes the effective portion 21 of the spectral probe for blood vessel diagnosis 10 to be pressed against the internal wall on the opposite side of the blood vessel 38. Because the measurement window 22 of the Raman probe is disposed on the opposite side of the balloon 23 with respect to the effective portion 21, consequently the measurement window 22 is pressed against the internal wall of the blood vessel 38. At this time, the balloon 23 does not occupy the entire space of the blood vessel 38 but leaves a gap between the balloon 23 as well as the spectral probe for blood vessel diagnosis 10 and the internal wall of the blood vessel. Therefore, the blood stream is ensured during measurement, and the burden on the human body is reduced. After the probe tip is fixed, the operator irradiates the blood vessel wall with excitation light using the Raman probe spectroscopy system 31, and then measures the Raman scattering spectra.

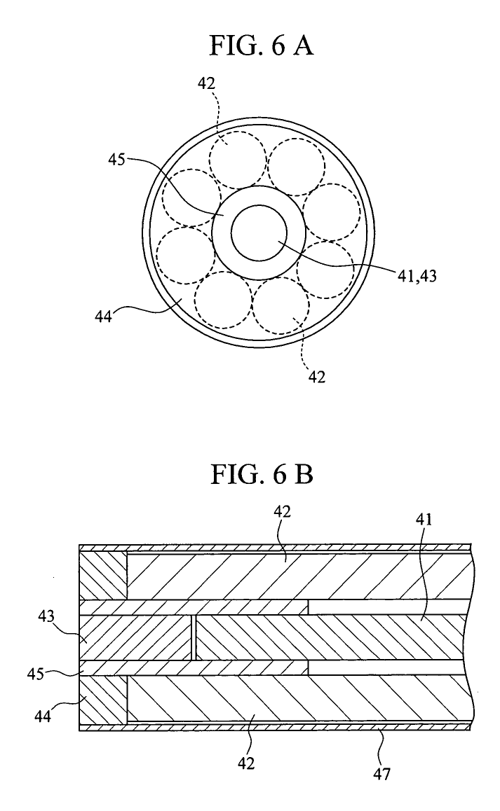

FIGS. 6A and 6B show a schematic view of the structure of the tip of the Raman probe portion. FIG. 6A shows a cross-sectional view, and FIG. 6B shows a longitudinal sectional view.

The Raman probe of the embodiment comprises a single excitation-light optical fiber 41 disposed in the center and eight light-receiving optical fibers 42 disposed such that the central optical fiber is surrounded thereby. A bandpass filter 43 that transmits only the excitation wavelength irradiated by the excitation light source 32 is mounted at the tip of the excitation-light optical fiber 41. An edge filter (long-wavelength transmitting filter) 44 for blocking the excitation wavelength and yet transmitting Raman scattered light irradiated by a sample is mounted at the ends of the light-receiving optical fibers 42. A stainless-steel pipe 45 is mounted at the tip of the excitation-light optical fiber 41 for blocking the transmission of light between the excitation-light optical fiber 41 and the light-receiving optical fibers 42, such that the excitation light emitted by the excitation-light optical fiber 41 will not directly enter the light-receiving optical fibers 42. When measuring an anti-Stokes line as Raman scattered light, whose wavelength is shorter than the excitation wavelength, the edge filter may be comprised of a short-wavelength transmitting filter that blocks the excitation wavelength but transmits wavelengths shorter than the excitation wavelength. The edge filter 44 is attached to the ends of the light-receiving optical fibers 42 with an adhesive agent such as glass resin. The side surfaces of the tip are covered with an outer covering 47 made of a stainless-steel pipe or resin film.

A stainless-steel pipe with an external diameter of 200 μm and an internal diameter of 130 μm was employed as the pipe 45 mounted at the tip of the excitation-light optical fiber 41. It is not desirable if the pipe were made of plastic, polyimide, or the like, because that would not only cause fluorescence or Raman scattering due to the excitation light, but would also cause optical leaks into the light-receiving optical fibers and produce crosstalk. While the optical fibers having the same diameter are employed for both the excitation-light optical fiber and the light-receiving optical fibers in the illustrated example, the diameter of the single excitation-light optical fiber may be larger than the diameter of the light-receiving optical fibers. Alternatively, a plurality of excitation-light optical fibers may be bundled together and inserted into the pipe 45.

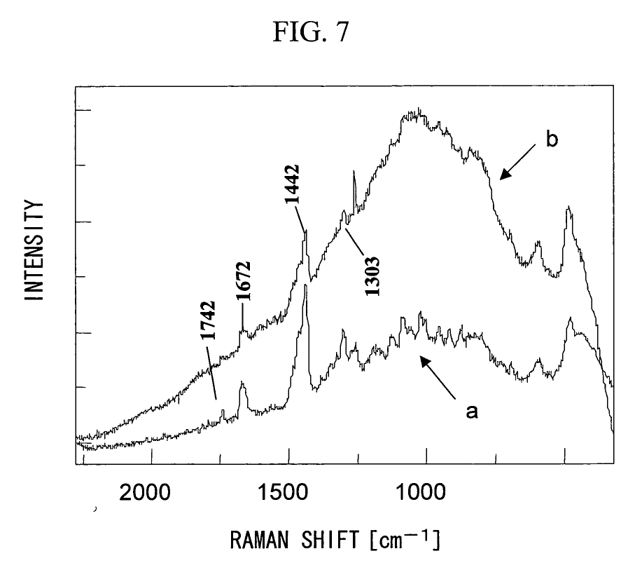

Next, an example of measurement using the spectral probe for blood vessel diagnosis of the embodiment will be described. A Raman scattering measurement was carried out on a blood vessel of a rabbit, which had been bred such that cholesterol would accumulate in its blood vessels. FIG. 7 shows the results. In FIG. 7, a spectrum a shows the Raman scattering spectrum of cholesterol oleate measured with the probe of the invention. A spectrum b shows the Raman scattering spectrum obtained by measuring the blood vessel of the rabbit with the use of the spectral probe for blood vessel diagnosis. While spectrum b contains fluorescence from biological cell tissues, in contrast to spectrum a, because the individual wavenumber portions correspond with one another, accumulation of cholesterol in the blood vessel was confirmed.

In accordance with the embodiment, images of shape and color information of an affected area can be obtained from the endoscope portion, and molecular-level information of the affected area can be obtained from the Raman probe portion. Based on such information, it is also possible to clarify the correlation between the shape/color information and the molecular information in the blood vessel, which has been unachievable.

Hereafter, an embodiment of the spectral probe for blood vessel diagnosis incorporating an ultrasonic probe portion instead of the endoscope portion will be described.

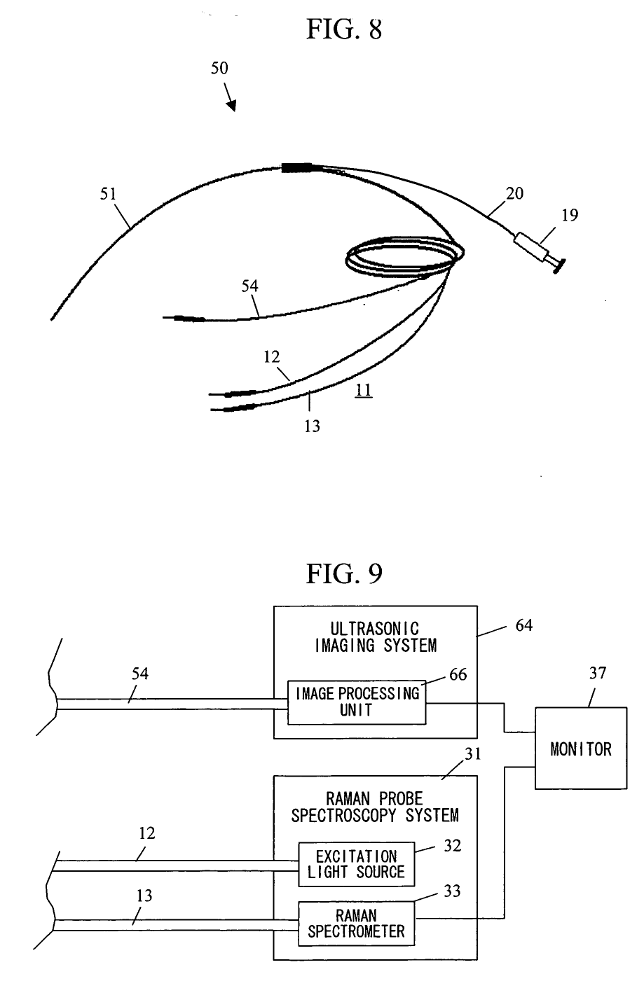

FIG. 8 shows an overall view of another embodiment of the spectral probe for blood vessel diagnosis of the invention. The embodiment comprises an ultrasonic probe portion as an image acquisition means instead of the endoscope portion.

A spectral probe for blood vessel diagnosis 50 of the embodiment comprises a Raman probe portion 11 comprising an excitation-light optical fiber 12 and a light-receiving optical fiber 13, an ultrasonic probe portion 54 as a blood vessel image acquisition means, and a tube 20 to which a balloon syringe 19 is connected. The overall length of the spectral probe for blood vessel diagnosis 50 is 3 m, and the length of an effective portion 51 that can be inserted into a blood vessel is 1.5 m. The diameter of the effective portion 51 is 2 mm in order to enable measurement in the artery. Because a blood vessel image is obtained by ultrasonic waves, there is no need for the means for introducing a blood-removing solution, which has been necessary when employing the endoscope portion.

FIG. 9 shows a diagram of the connection between the spectral probe for blood vessel diagnosis of the embodiment and the outside. The excitation-light optical fiber 12 and the light-receiving optical fiber 13 of the Raman probe portion 11 are connected to an excitation light source 32 and a Raman spectrometer 33, respectively, of a Raman probe spectroscopy system 31. The ultrasonic probe portion 54 is connected to an image processing unit 66 of an ultrasonic imaging system 64. Information on density distribution in a cross section (depth direction) at different locations of blood vessel walls is acquired by moving the ultrasonic probe along the blood vessel wall. An ultrasonic transducer is disposed in the center of the tip portion. On a monitor 37, Raman scattering spectra of an affected area obtained by the Raman spectrometer 33 is also displayed.

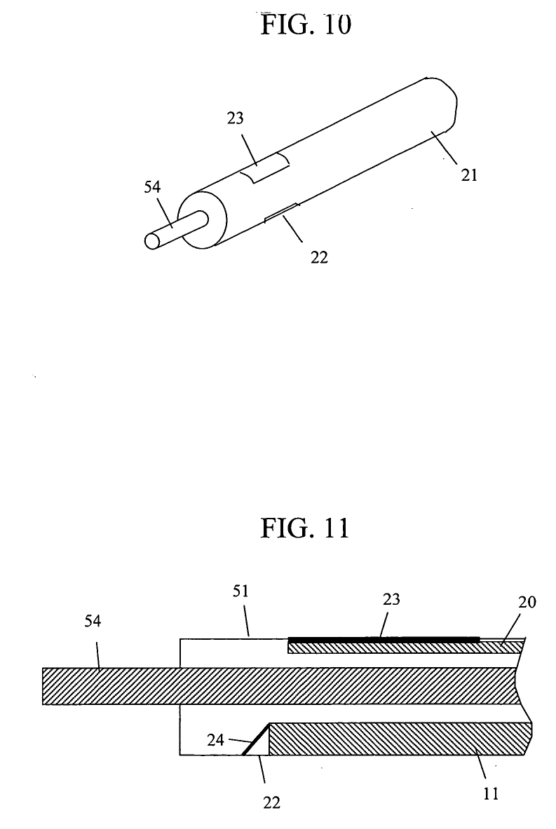

FIG. 10 shows a schematic perspective view of the structure of the effective portion tip of the spectral probe for blood vessel diagnosis of the embodiment. FIG. 11 shows its schematic cross-section.

The tip of the ultrasonic probe portion 54 protrudes from the tip of the effective portion 51 that is inserted into a blood vessel. The sensor portion of the ultrasonic probe portion 54 is provided in the protruded tip portion. This is because if the sensor portion were provided in the effective portion 51 of the probe, ultrasonic information about the blood vessel walls would not be obtained. A Raman measurement window 22 is provided on the side of the effective portion 51, and the balloon 23 is provided on the side of the effective portion opposite to the Raman measurement window 22. When the balloon 23 is inflated, the normal saline solution is injected from the balloon syringe 19 via the tube 20. As shown in FIG. 11, excitation light from the Raman probe 11 is reflected by the mirror 24 provided inside the effective portion 51, and shone against the blood vessel walls through the Raman measurement window 22. The Raman scattered light produced by the blood vessel walls as they are irradiated with the excitation light passes through the Raman measurement window 22, is reflected by the mirror 24, and then introduced into the Raman probe 11.

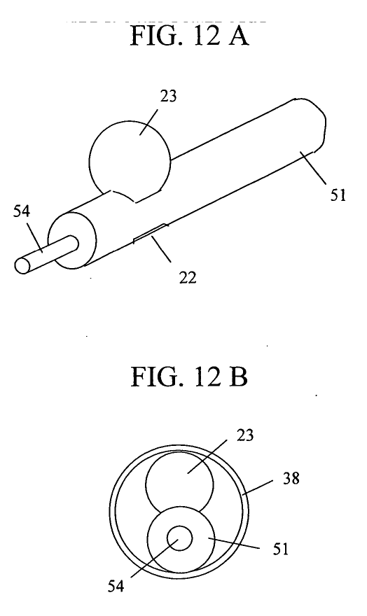

FIGS. 12A and 12B show a schematic diagram of the balloon being inflated in a blood vessel during measurement. FIG. 12A shows a perspective view of the tip of the effective portion of the spectral probe for blood vessel diagnosis, and FIG. 12B shows a schematic cross-sectional view of the blood vessel viewed from the probe tip.

The balloon 23 has the function of fixing the effective portion 51 of the spectral probe for blood vessel diagnosis 50 at a desired location in a blood vessel 38, and the function of bringing the measurement window 22 of the Raman probe into contact with an affected area. Information on the density distribution in a depth direction of the blood vessel wall is obtained by an ultrasonic image. The operator determines a measurement location while monitoring an ultrasonic image of the blood vessel obtained by the ultrasonic probe portion 54 on the monitor 37. Because the ultrasonic image is not subject to the interference of blood, there is no need to remove blood using the normal saline solution or the like for obtaining an image.

Once the measurement location is determined, the normal saline solution is injected into the balloon 23 from the balloon syringe 19, so as to inflate the balloon 23 for fixing the probe tip at the measurement location. As shown in FIG. 12B, the balloon 23 comes into contact with the internal wall of the blood vessel 38, and causes the effective portion 21 of the spectral probe for blood vessel diagnosis 50 to be pressed against the opposite internal wall of the blood vessel 38. Because the measurement window 22 of the Raman probe is provided on the opposite side of the balloon 23 with respect to the effective portion 21, the measurement window 22 is pressed against the internal wall of the blood vessel 38. At this time, the balloon 23 does not occupy the entire space of the blood vessel 38 but leaves a gap between the balloon 23 as well as the spectral probe for blood vessel diagnosis 50 and the internal wall of the blood vessel. Therefore, the blood stream is ensured during measurement, and the burden on the human body is reduced. In this state, the operator measures the Raman scattering spectra of the blood vessel wall, using the Raman probe spectroscopy system 31.

In accordance with the foregoing embodiment, information on the cross-sectional shape (vessel diameter) of the affected area and density differences is obtained with ultrasonic waves, while molecular-level information is obtained with the Raman probe. Therefore, new information can be obtained from the correlation between these data.

What is claimed is:

1. A spectral probe for blood vessel diagnosis comprising:
a long member for insertion into a blood vessel;
a light-transmitting window provided in a side wall of the tip of the long member;
a balloon provided in the side wall of the tip of the long member opposite to the light-transmitting window;
a Raman probe portion comprising an optical fiber for guiding light from a light source to the light-transmitting window, and an optical fiber for guiding Raman scattered light that enters the long member via the light-transmitting window to the outside;
a tube for introducing a fluid into the balloon;
an endoscope portion comprising an optical fiber for guiding illumination light to the tip of the long member, and an image fiber; and
a tube for injecting normal saline solution into an area that is irradiated with the illumination light from the endoscope portion.

2. The spectral probe for blood vessel diagnosis according to claim 1, wherein, when the balloon is inflated, a gap is formed between the inflated balloon and the internal walls of the blood vessel.

3. A spectral probe for blood vessel diagnosis comprising:
a long member for insertion into a blood vessel;
a light-transmitting window provided in a side wall of the tip of the long member;
a balloon provided in the side wall of the tip of the long member opposite to the light-transmitting window;
a Raman probe portion comprising an optical fiber for guiding light from a light source to the light-transmitting window, and an optical fiber for guiding Raman scattered light that enters the long member via the light-transmitting window to the outside;
a tube for introducing a fluid into the balloon; and
an ultrasonic probe for obtaining an ultrasonic image of an area at the tip of the long member.

4. The spectral probe for blood vessel diagnosis according to claim 3, wherein, when the balloon is inflated, a gap is formed between the inflated balloon and the internal walls of the blood vessel.
